(19) United States
(12) Patent Application Publication
(10) Pub. No.: US 2026/0083473 A1
(43) Pub. Date: Mar. 26, 2026

(54) PENETRATING ROLLER FOR ENDOVASCULAR USE AND RELATED METHODS

(71) Applicant: BARD PERIPHERAL VASCULAR, INC., Franklin Lakes, NJ (US)

(72) Inventors: Matthew R. CASIRARO, Tempe, AZ (US); Andrzej J. CHANDUSZKO, Chandler, AZ (US)

(21) Appl. No.: 19/113,584

(22) PCT Filed: Sep. 27, 2022
(86) PCT No.: PCT/US2022/044903

§ 371 Date: Mar. 20, 2025

(51) Int. Cl. A61B 17/3207; A61B 17/00; A61B 34/00

(52) U.S. Cl. CPC A61B 17/320725; A61B 34/73; A61B 2017/00867

(57) ABSTRACT

An apparatus is for penetrating a vessel inner wall, such as to treat a lesion in an artery or to achieve closure in a vein, as examples. The apparatus includes a first shaft (12) adapted for passing through a sheath (26) adapted for insertion into the vessel to a 2024/072378 location in need of treatment. The first shaft supports a first roller (14) adapted to penetrate the inner wall of the vessel. Optionally, the apparatus may include a second shaft supporting a second roller adapted to penetrate the vessel wall. The first or second rollers may be further adapted to rotate about an axis transverse to a longitudinal axis of the first or second shafts, respectively. Related methods are also disclosed.

### BACKGROUND

Scoring catheters often employ one or more wires in connection with an inflatable balloon to apply a focal force against an inner wall of a vessel, which may comprise a calcified lesion. A limitation of this approach is the limited scoring effect achieved in terms of the depth. Consequently, a drug, such as an anti-proliferative agent, may remain unable to penetrate inner calcium layers in the lesion (in particular, the medial layer, which is notoriously calcified in diabetic patients having peripheral artery disease). The use of a scoring wire also requires precise sizing of an associated balloon based on the lesion, and sometimes limits the effectiveness to a particular area corresponding to the dimension of the wire, which is typically quite narrow.

Accordingly, a need exists for an approach to penetrating a lesion in a vessel that can achieve the benefit of providing for enhanced penetration and without limiting the use to a particular area based on the size of a scoring wire or associated balloon.

### SUMMARY

An object of the disclosure is to provide an apparatus for penetrating an inner wall of a vessel that can achieve the benefit of providing for enhanced penetration, without limiting the use to a particular area based on the size of a scoring wire or associated balloon, thereby allowing for use on an unlimited length of vessel.

According to one aspect of the disclosure, the apparatus for penetrating an inner wall of a vessel comprises a sheath adapted for insertion in the vessel and a first shaft adapted for passing through the sheath. The first shaft supports a first roller adapted to penetrate the inner wall. The first roller may be further adapted to rotate about an axis transverse to a longitudinal axis of the first shaft.

In one embodiment, the first roller comprises a drum supporting a plurality of radially extending projections. The drum and the plurality of radially extending projections may comprise a unitary structure.

The first shaft includes a distal portion adapted for biasing the first roller toward the inner wall. The distal portion may comprise a shape memory material. The apparatus may further include a balloon for urging the distal portion to bias the first roller toward the inner wall. Alternatively or additionally, the distal portion comprises opposing magnets for urging the first roller toward the inner wall, or a spring could be used.

The apparatus may further include a second shaft supporting a second roller adapted for rotation about an axis transverse to a longitudinal axis of the second shaft. A balloon may be provided for urging the first and second rollers apart from each other. Alternatively or additionally, the first and second shafts comprise opposing magnets for urging the first and second rollers apart.

In any disclosed embodiment, the apparatus may be used in combination with a balloon catheter including a treatment agent for delivery to the inner wall.

According to a further aspect of the disclosure, an apparatus for penetrating an inner wall of a vessel is provided. The apparatus comprises a first shaft including a distal portion supporting a first roller adapted to penetrate the inner wall. The distal portion is adapted for moving from a retracted position relative to the first shaft to a deployed position for urging the first roller toward the inner wall. The first roller may be further adapted to rotate about an axis transverse to a longitudinal axis of the first shaft.

In one embodiment, the apparatus further includes a sheath for receiving the first shaft. The first roller may comprise a drum supporting a plurality of radially extending projections. The drum and the plurality of radially extending projections may comprise a unitary structure.

The distal portion of the shaft may comprise a shape memory material. The apparatus may further include a balloon for urging the distal portion to bias the first roller toward the inner wall. Alternatively or additionally, the distal portion comprises opposing magnets for urging the distal portion to bias the roller toward the inner wall.

The apparatus may further include a second shaft supporting a second roller adapted to penetrate the inner wall. The second roller is further adapted to rotate about an axis transverse to a longitudinal axis of the second shaft. A balloon may be provided for urging the first and second rollers apart from each other. Alternatively or additionally, the first and second shafts may comprise opposing magnets for urging the first and second rollers apart.

Still a further aspect of the disclosure pertains to a method of penetrating a inner wall in a vessel. The method comprises engaging the inner wall with a first roller adapted to penetrate the inner wall. The first roller may be further adapted to rotate about an axis transverse to an axis of a shaft supporting the first roller.

In one embodiment, the engaging step comprises biasing the first roller toward the inner wall. The method may further include the step of providing the shaft with a second roller adapted to treat the inner wall, and further adapted for rotating about the transverse axis. The method may also involve the step of applying a treatment agent to the inner wall, which may be completed during or after the engaging step.

### BRIEF DESCRIPTION OF THE DRAWINGS

The above and further advantages of the disclosure may be better understood by referring to the following description in conjunction with the accompanying drawings in which:

FIG. 1 illustrates a side view of an apparatus for penetrating an inner wall in a vessel.

FIG. 1A is an end view of the apparatus located in a delivery sheath.

FIG. 2 is a schematic view of the sheath including the apparatus positioned in a vessel for treatment.

FIG. 3 illustrates deployment of the apparatus from the sheath.

FIG. 4 illustrates deployment of the apparatus for engaging the vessel.

FIG. 5 is a cross-section taken along line 5-5 of FIG. 4.

FIG. 6 illustrates an alternative embodiment of the apparatus.

FIGS. 7 and 7A are side and rear perspective views of the alternative embodiment of the apparatus of FIG. 6 in a deployed configuration.

FIG. 7B is a cross-sectional view taken along like 7B-7B of FIG. 7.

FIG. 8 illustrates an alternative embodiment of a penetrating roller.

FIG. 9 illustrates another alternative embodiment of a penetrating roller.

FIG. 10 is a schematic view illustrating the application of a treatment to a vessel following the use of the penetrating roller.

The dimensions of some of the elements may be exaggerated relative to other elements for clarity or several physical components may be included in one functional block or element. Further, sometimes reference numerals may be repeated among the drawings to indicate corresponding or analogous elements. Moreover, some of the items depicted in the drawings may be combined into a single function.

### DETAILED DESCRIPTION

In the following detailed description, numerous specific details are set forth to provide a thorough understanding of the present invention. The disclosed embodiments may be practiced without these specific details. In other instances, well-known methods, procedures, components, or structures may not have been described in detail so as not to obscure the present invention.

The principles and operation of the apparatus and methods of the disclosure may be better understood with reference to the drawings and accompanying descriptions. The invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

Certain features of the invention that are, for clarity, described in the context of separate embodiments, may also be provided in combination in a single embodiment. Conversely, various features of the invention that are, for brevity, described in the context of a single embodiment, may also be provided separately or in any suitable sub-combination.

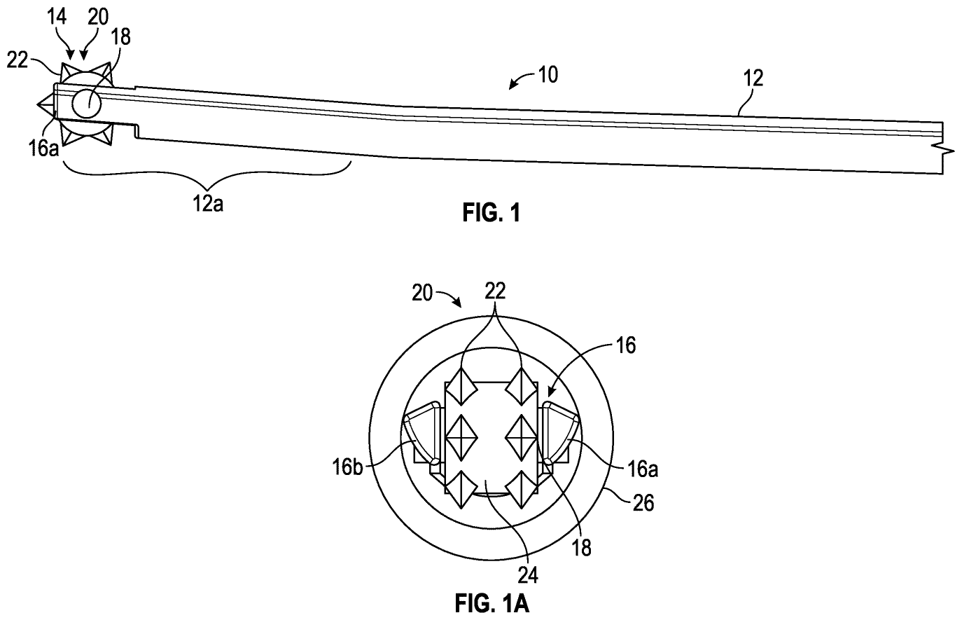

With reference to FIG. 1, an apparatus 10 for penetrating an inner wall in a vessel is shown, which includes an elongated shaft 12 having a roller 14 adapted for penetrating the vessel wall, which as noted below may comprise a lesion. As perhaps best understood from FIG. 1A, the shaft 12 includes a yoke 16 comprising a pair of spaced apart, elongated arms 16a, 16b for supporting the roller 14 positioned there between. Specifically, the yoke 16 supports a pin 18 about which the roller 14 may rotate, with the pin extending transversely to a longitudinal axis of the shaft 12 and being fixed to the arms 16a, 16b.

As can be further understood from FIGS. 1 and 1A, the roller 14 includes sharp projections 20, or “sharps,” adapted for perforating tissue forming the vessel wall or an associated lesion forming part of the wall. In this embodiment, the projections 20 extend radially in a direction corresponding to a diameter of the associated drum 24. In one example, the projections 20 comprise two spaced rows of circumferentially spaced spikes 22, which may be connected directly to a drum 24 forming the roller 14. As such, a unitary structure is formed. However, it is possible to make the projections 20 removable, or part of a separate sleeve or like structure that may be removably attached to the drum 24 (which may allow for any desired modification to the amount or depth of penetration achieved for a given treatment situation). Alternatively, the drum 24 itself can simply be replaced. The projections 20 and drum 24 may be fabricated of stainless steel or similar material (400 series stainless steels hold a sharper edge whereas 300 series are cheaper to manufacture, but either would work for this application), and thus would be visible under fluoroscopy. The diameter of the roller 14 is less than the longitudinal dimension of the arms 16a, 16b forming the yoke 16 so as to allow for free rotation without interference by the face of the shaft 12 between these arms opposing the roller.

As can also be understood, the distal end portion 12a of the shaft 12 may be normally skewed or biased in a vertical direction a small amount (e.g., 5 -15 degrees). Thus, when a proximal portion of the shaft 12 is passing along and alighting from an associated sheath 26 in contact with a surface thereof, the spikes 22 may be maintained in an intermediate position relative to the diameter of the sheath 26. Consequently, the spikes 22 do not contact the interior of the sheath 26 or the vessel, at least until such contact is desired.

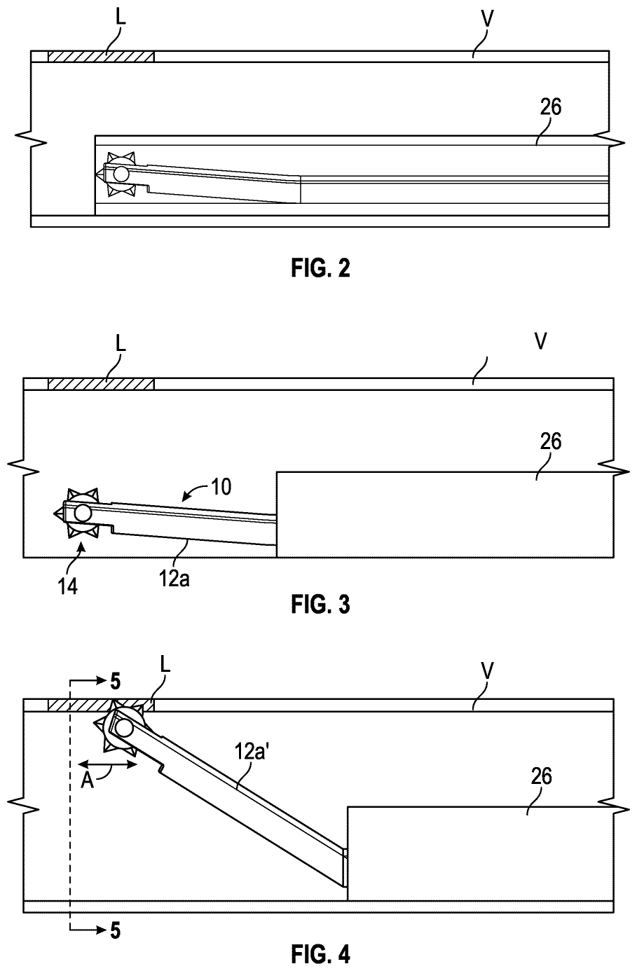

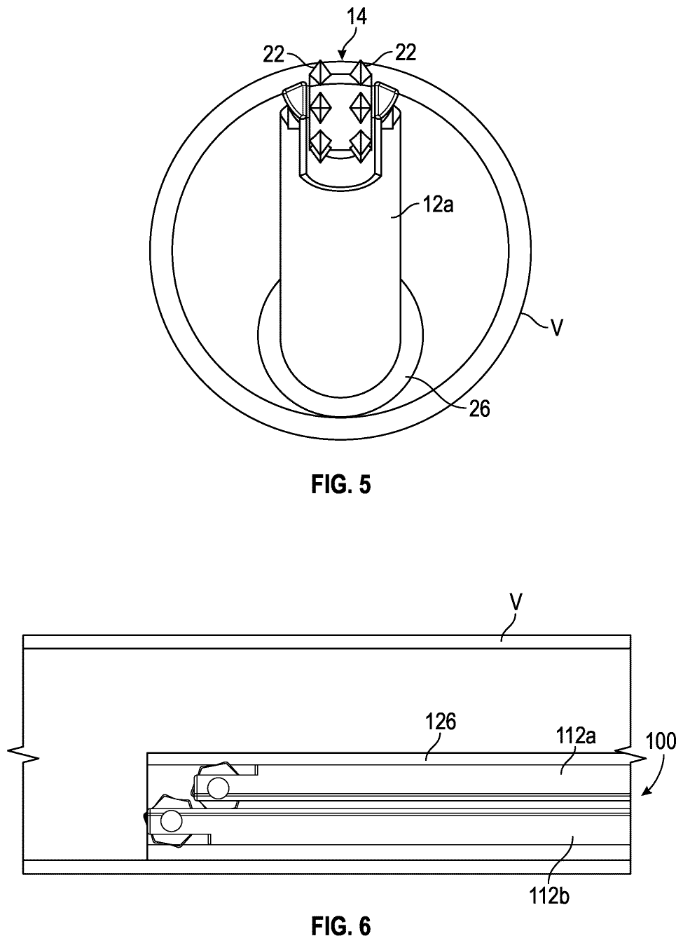

In use, and with reference to FIGS. 2, 3, and 4, the apparatus 10 may be advanced within a vessel V to a location where a treatment is desired, such as penetrating a lesion L forming part of the vessel inner wall for the application of a treatment agent, for example. During the transport, the apparatus 10 may be fully contained within the sheath 26, and then exposed by advancing it, withdrawing the sheath, or both, as shown in FIG. 3. The distal end portion 12a of the shaft 12 may then be biased toward the inner wall of the vessel V (note 12a′ which is at an angle of approximately 45 degrees relative to the remainder of the shaft 12) to move the roller 14 from a retracted position to a deployed position, thus urging the roller toward the vessel inner wall. This causes engagement of the inner wall and, in particular, a lesion L in this example, with the roller 14 and, in particular, the spikes 22, as shown in FIGS. 4 and 5. This may be achieved by forming at least the distal end portion 12a of a shape memory material, such as Nitinol, which upon being activated such as by a temperature change, causes the desired positioning (and which position can be reversed when desired). As noted below, this movement may also be achieved by using an expandable element, such as a balloon, opposing magnets, a spring, or any combination of the foregoing.

As can be understood, in this position, the roller 14 may be moved to and fro within the vessel V, while the roller 14 and its spikes 22 engage the interior thereof, such as along a lesion L. In view of the ability of the roller 14 to rotate continuously, the apparatus 10 thus affords the ability to penetrate an elongated lesion or section of a vessel by longitudinal movement of the roller 14 (including possibly in a repeated manner), as indicated by arrow A, and also possibly with rotation of the shaft 12 to contact different portions of the vessel inner wall. This is contrasted with the past approach to scoring a lesion using one or more thin wires to achieve a single linear point of contact, or requiring the use of a particular fixed length of a scoring balloon to effect limited penetration. Moreover, the ability to control the depth of penetration into the vessel wall via the dimensions of the spikes 22 or the biasing of the roller 14 affords further advantages, such as by allowing for the ready penetration of an intermediate calcified layer of a lesion in one particular example (but could also be used in situations where the vessel wall lacks a lesion, such as a vein where achieving closure is desired).

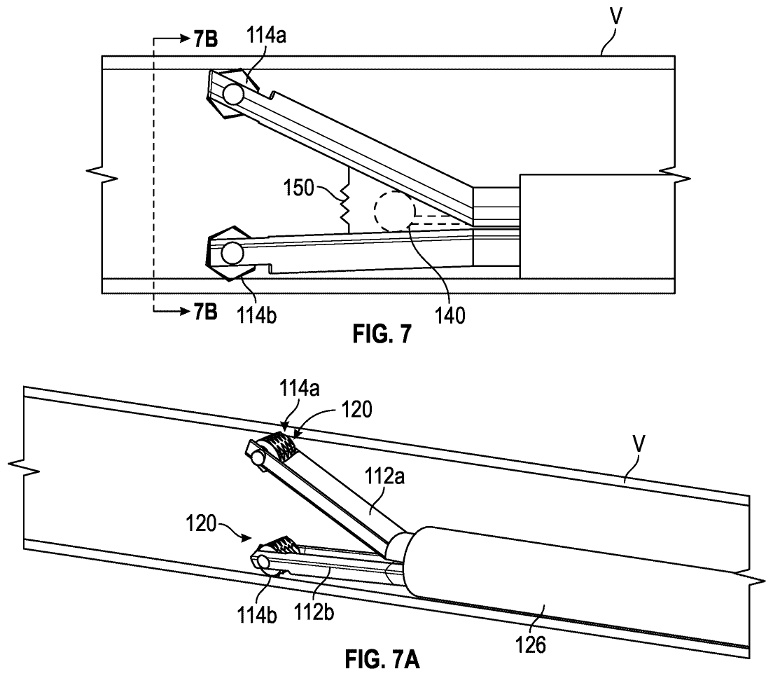

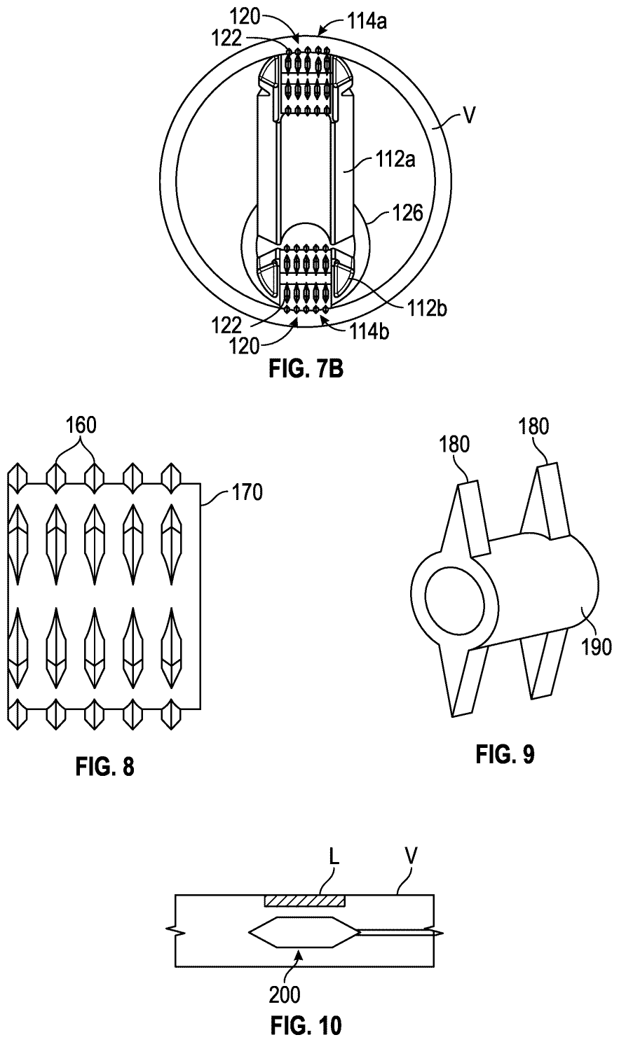

An alternative embodiment of the apparatus 100 is shown in FIGS. 6, 7, 7A, and 7B. In this version, two shafts 112a, 112b are provided. The shafts 112a, 112b may be sized to fit into a single sheath 126 together, and each with a penetrating roller 114a, 114b. The rollers 114a, 114b may comprise projections 120, such as spikes 122, and thus adapt the rollers 114a, 114b for penetrating the vessel. The rollers 114a, 114b may also be adapted to rotate about an axis transverse to a longitudinal axis of the respective shaft 112a, 112b, but in a different horizontal plane.

In use, and similar to the first embodiment described above, the shafts 112a, 112b may be deployed from the sheath 126. Once deployed, the shafts 112a, 112b may be biased apart to cause rollers 114a, 114b to concurrently engage different portions of the interior of the vessel V, as shown in FIGS. 7 and 7A. The biasing may be achieved by using shape memory materials, as noted, or may be achieved using alternative means. For example, as shown in FIG. 7, an inflatable balloon 140 may be positioned between the shafts 112a, 112b (or more particularly the distal end portions, which may comprise spring steel) and expanded, such as via inflation fluid supplied via a catheter extending through the sheath 126 (such as in a lumen or space formed between the shafts 112a, 112b). This expansion force causes one or both of the rollers 114a, 114b to bias outwardly (in the illustrated embodiment, only shaft 112a moves relative to shaft 112b, but both could be adapted to move, or the arrangement may be reversed). The shafts 112a, 112b may also be magnetized with opposite poles, such that upon aligning from the sheath 126, the repelling force causes the desired biasing to occur so that the rollers 114a, 114b achieve engagement with a target area, such as a lesion. Instead of a balloon, one or more springs could also be used, such as a leaf or compression spring as illustrated schematically in FIG. 7. Any combination of the foregoing structures could be used.

Referring to FIGS. 8 and 9, it can be understood that the projections 20, 120 may take various shapes and forms. For example, in FIG. 8 (and also FIGS. 7A and 7B), the projections are in the form of circumferentially spaced blades 160 arranged in rows, such as the five shown, but it could be fewer or more. The associated drum 170 may also be cylindrical or polygonal in cross-section, with “flats” supporting the projections. FIG. 9 illustrates that the projections may take the form of elongated needles or spikes 180, which may be provided in one or more spaced pairs (circumferentially or axially spaced, or both as shown) around a tubular drum 190.

Once the penetration using the roller 14, 114a, 114b is completed, a treatment may be provided. As shown in FIG. 10, for example, this may be achieved by advancing a drug-coated balloon 200 to the treatment area, and expanding the same to achieve contact with the treatment area, such as a lesion. As can be appreciated, the perforations formed as a result of the contact with the roller 14, 114a, 114b thus facilitate having the treatment agent or drug penetrate into the lesion and thus achieve an enhanced treatment effect. It is also possible for the projections 20 to be coated or otherwise provided with a treatment agent to achieve delivery immediately upon penetration.

Summarizing, this disclosure may be considered to relate to the following items:
1. An apparatus for penetrating an inner wall of a vessel, comprising:
a sheath adapted for insertion in the vessel; and
a first shaft adapted for passing through the sheath, the first shaft supporting a first roller adapted to penetrate the inner wall of the vessel.
2. The apparatus of item 1, wherein the first roller is further adapted to rotate about an axis transverse to a longitudinal axis of the first shaft.
3. The apparatus of item 1 or 2, wherein the first roller comprises a drum supporting a plurality of radially extending projections.
4. The apparatus of item 3, wherein the drum and the plurality of radially extending projections comprise a unitary structure.
5. The apparatus of any of items 1-4, wherein the first shaft comprises a distal portion adapted for biasing the first roller toward the inner wall.
6. The apparatus of item 5, wherein the distal portion comprises a shape memory material.
7. The apparatus of item 5 or 6, further including a balloon for urging the distal portion to bias the first roller toward the inner wall.
8. The apparatus of item 5, 6 or 7, wherein the distal portion comprises opposing magnets for urging the first roller toward the inner wall.
9. The apparatus of any of items 5 to 8, wherein the distal portion comprises a spring for urging the first roller toward the inner wall of the vessel.
10. The apparatus of any of items 1 to 5, further including a second shaft supporting a second roller adapted for rotation about an axis transverse to a longitudinal axis of the second shaft.
11. The apparatus of item 10, further including a balloon for urging the first and second rollers apart from each other.
12. The apparatus of item 10 or item 11, wherein the first and second shafts comprise opposing magnets for urging the first and second rollers apart.
13. The apparatus of any of items 10-12, further including a spring for urging the first and second rollers apart.
14. The apparatus of any of items 1-13, in combination with a balloon catheter including a treatment agent for delivery to the inner wall of the vessel.

15. An apparatus for penetrating an inner wall of a vessel, comprising:
a first shaft including a distal portion supporting a first roller adapted to penetrate the inner wall, the distal portion of the first shaft adapted for moving from a retracted position to a deployed position for urging the first roller toward the inner wall.
16. The apparatus of item 15, further including a sheath for receiving the first shaft.
17. The apparatus of item 15 or 16, wherein the first roller is further adapted to rotate about an axis transverse to a longitudinal axis of the first shaft.
18. The apparatus of any of items 15 to 17, wherein the first roller comprises a drum supporting a plurality of radially extending projections.
19. The apparatus of item 18, wherein the drum and the plurality of radially extending projections comprise a unitary structure.
20. The apparatus of any of items 15 to 19, wherein the distal portion comprises a shape memory material.
21 The apparatus of any of items 15 to 20, further including a balloon for urging the distal portion to bias the first roller toward the inner wall of the vessel.
22. The apparatus of any of items 15 to 21, wherein the distal portion comprises opposing magnets for urging the distal portion to bias the roller toward the inner wall of the vessel.
23. The apparatus of any of items 15-22, wherein the distal portion comprises a spring for urging the first roller toward the inner wall of the vessel.
24. The apparatus of any of items 15 to 20, further including a second shaft supporting a second roller adapted to penetrate the lesion, the second roller further adapted to rotate about an axis transverse to a longitudinal axis of the second shaft.
25. The apparatus of item 24, further including a balloon for urging the first and second rollers apart from each other.
26. The apparatus of item 24 or item 25, wherein the first and second shafts comprise opposing magnets for urging the first and second rollers apart.
27. The apparatus of any of items 24-26, further including a spring for urging the first and second rollers apart.
28. The apparatus of any of claims 15 to 27, in combination with a balloon catheter including a treatment agent for delivery to the inner wall of the vessel.
29. A method of penetrating an inner wall of a vessel, comprising: engaging the inner wall with a first roller adapted to penetrate the inner wall of the vessel.
30. The method of item 29, wherein the first roller is further adapted to rotate about an axis transverse to a longitudinal axis of a shaft supporting the first roller during the engaging step.
31. The method of item 29 or item 30, wherein the engaging step comprises biasing the first roller toward the lesion.
32. The method of any of items 29-31, further including the step of providing the shaft with a second roller adapted to penetrate the inner wall, and further adapted for rotating about the transverse axis.
33. The method of any of items 29-32, further including the step of applying a treatment agent to the inner wall.
34. The method of claim 33, wherein the applying step is completed after the engaging step.

As used herein, the following terms have the following meanings:
“A”, “an”, and “the” as used herein refers to both singular and plural referents unless the context clearly dictates otherwise. By way of example, “a compartment” refers to one or more than one compartment.
“About,” “substantially,” or “approximately,” as used herein referring to a measurable value, such as a parameter, an amount, a temporal duration, and the like, is meant to encompass variations of +/−20% or less, including +/−10% or less, +/−5% or less, +/−1% or less, and +/−0.1% or less of and from the specified value, in so far such variations are appropriate to perform in the disclosed invention. However, it is to be understood that the value to which such modifiers refer is itself also specifically disclosed.
“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Although the invention has been described in conjunction with specific embodiments, many alternatives, modifications, and variations will be apparent to those skilled in the art. Accordingly, it embraces all such alternatives, modifications, and variations that fall within the spirit and scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, or identification of any reference in this application shall not be construed as an admission that such reference is available as prior art to the present disclosure.

1. An apparatus for penetrating an inner wall of a vessel, comprising:
a sheath adapted for insertion in the vessel; and
a first shaft adapted for passing through the sheath, the first shaft supporting a first roller adapted to penetrate the inner wall of the vessel.

2. The apparatus of claim 1, wherein the first roller is further adapted to rotate about an axis transverse to a longitudinal axis of the first shaft.

3. The apparatus of claim 1, wherein the first roller comprises a drum supporting a plurality of radially extending projections.

4. The apparatus of claim 3, wherein the drum and the plurality of radially extending projections forma unitary structure.

5. The apparatus of claim 1, wherein the first shaft comprises a distal portion adapted for biasing the first roller toward an inner wall of the vessel.

6. The apparatus of claim 5, wherein the distal portion comprises a shape memory material.

7. The apparatus of claim 5, further including a balloon for urging the distal portion to bias the first roller toward the inner wall of the vessel.

8. The apparatus of claim 5, wherein the distal portion comprises opposing magnets for urging the first roller toward the inner wall of the vessel.

9. The apparatus of claim 5, wherein the distal portion comprises a spring for urging the first roller toward the inner wall of the vessel.

10. The apparatus of claim 1, further including a second shaft supporting a second roller adapted for rotation about an axis transverse to a longitudinal axis of the second shaft.

11. The apparatus of claim 10, further including a balloon for urging the first and second rollers apart from each other.

12. The apparatus of claim 10, wherein the first and second shafts comprise opposing magnets for urging the first and second rollers apart.

13. The apparatus of claim 10, further including a spring for urging the first and second rollers apart.

14. The apparatus of claim 1, in combination with a balloon catheter including a treatment agent for delivery to the lesion.

15. An apparatus for penetrating an inner wall of a vessel, comprising:
a first shaft including a distal portion supporting a first roller adapted to penetrate the inner wall, the distal portion of the first shaft adapted for moving from a retracted position to a deployed position for urging the first roller toward the inner wall.

16. The apparatus of claim 15, further including a sheath for receiving the first shaft.

17. The apparatus of claim 15, wherein the first roller is further adapted to rotate about an axis transverse to a longitudinal axis of the first shaft.

18. The apparatus of claim 15, wherein the first roller comprises a drum supporting a plurality of radially extending projections.

19. The apparatus of claim 18, wherein the drum and the plurality of radially extending projections form a unitary structure.

20.-23. (canceled)

24. The apparatus of claim 15, further including a second shaft supporting a second roller adapted to penetrate the vessel, the second roller further adapted to rotate about an axis transverse to a longitudinal axis of the second shaft.

25.-34. (canceled)
